(19) United States
(12) Patent Application Publication
(10) Pub. No.: US 2018/0116565 A1
(43) Pub. Date: May 3, 2018

(54) Method and Device for Administering a Hearing Test

(71) Applicant: MEQ INC., Albany, CA (US)

(72) Inventors: Ofer RAZ, Albany, CA (US); Danny ARONSON, Tel Aviv (IL)

(21) Appl. No.: 15/571,873

(22) PCT Filed: May 4, 2016
(86) PCT No.: PCT/IL2016/000007

§ 371 Date: Nov. 6, 2017

Related U.S. Application Data
(60) Provisional application No. 62/157,990, filed on May 7, 2015.

(51) Int. Cl. A61B 5/12; A61B 5/00

(52) U.S. Cl. CPC A61B 5/123; A61B 5/486

(57) ABSTRACT

A method is provided for administering a hearing test to an individual. The method comprises the steps of: selecting at least one musical melody; determining a plurality of frequencies at which the at least one musical melody or part thereof will be played for the individual; executing instructions comprised in a computer program to perform steps for administering a hearing test to the individual, wherein the instructions are configured to enable the individual to listen to the at least one musical melody or part thereof at the determined frequencies and at varying intensities; and establishing hearing capabilities of the individual to whom the hearing test has been administered, based on that individual's reactions while listening to the at least one musical melody or part thereof at the determined frequencies and at varying intensities.

### TECHNICAL FIELD

The present disclosure relates to the field of audiology, and in particularly, to a method and device for administering a hearing test.

### BACKGROUND

Amongst the various tests that may be carried out to teat one's hearing, pure tone audiometry is the most common one. It evaluates the sensitivity of the hearing sense at different frequencies. A set of headphones is worn over the ears or foam earphones are placed in the ear canal, and the user being tested, is required to sit in a sound-proof test cubical and use the headphones which are connected to an audiometer. The audiometer produces tones at specific frequencies and levels for each ear, independently. The user (patient) indicates that he has heard the tone, by either raising his hand or by pressing a button.

Pure tone audiometry (“PTA”) is considered as the key hearing test used to identify hearing threshold levels of an individual (a user), enabling determination of the degree, type and configuration of deterioration in his hearing capabilities. PTA is a subjective, behavioural measurement of hearing threshold, as it relies on the user's responses to pure tones stimuli.

In a typical PTA hearing test, the results are provided in a form of an audiogram, i.e. a graphic representation of the results obtained during the individual's hearing test. Frequency, measured in Hertz (Hz), is plotted on the abscissa, generally in the range of from 250 Hz (0.25 kHz) up to 8000 Hz (8 kHz). The audiogram represents each frequency octave starting at 250 Hz. An octave is a doubling of frequency, so that a standard audiogram includes results obtained at 250 Hz, 500 Hz, 1000 Hz, 2000 Hz, 4000 Hz, and 8000 Hz. In many cases, especially when a large difference is measured between two octave frequencies, the inter-octaves frequencies will also be tested, such as at 750 Hz, 1500 Hz, 3000 Hz, and 6000 Hz.

The ordinate of the audiogram represents the intensity, plotted in decibels, of the hearing level (dB HL). Audiograms are generally arranged with lower intensities represented at the top and higher intensities represented at the bottom, increasing by 5 dB steps. Audiograms usually represent a range from −10 dB HL to 100 or 120 dB HL. Normal hearing is considered to be 20 dB HL or better.

When considering the term frequency in music, one refers to how often a sonic event happens in its entirety over the course of a specified time segment. For a sound to have a perceived frequency, it must be a periodic tone (repeated in time). Since the period of an event is the length of time which takes the event to occur, it is clear that the two concepts (periodicity and frequency) are related, if not equivalent.

Musicians usually talk about the frequency content of their music in terms of pitch, or sets of pitches called scales. Humans tend to recognize relative relationships, not just absolute physical values, and when they do, those relationships (especially in the aural domain) tend to be logarithmic. That is, human do not perceive the difference (subtraction) between two frequencies, but rather the ratio (division) thereof.

This means that it is much easier for most humans to hear or describe the relationship or ratio between two frequencies than to name the exact frequencies that they hear. In fact, for most of the people, the exact frequencies are not even very important. The common musical term for this is transposition: we hear the tune correctly, no matter what key it is played in. Following is a simple example. The difference to the human ears between 101 Hz and 100 Hz is much greater than the difference between 1,001 Hz and 1,000 Hz. We do not hear a change of 1 Hz in each of these two cases, instead we hear a change of 1,001/1,000 (=1.001) as compared with a much bigger change of 101/100 (=1.01).

Most humans tend to organize the frequency spectrum between 20 Hz and 20 kHz roughly into octaves, which means powers of 2. In other words, we perceive the same pitch difference between 100 Hz and 200 Hz as we do between 200 Hz and 400 Hz, 400 Hz and 800 Hz, and so on, where in each case, the ratio of the two frequencies is 2:1.

In almost all musical cultures, pitches are named not by their actual frequencies, but as general categories of frequencies in relationship to other frequencies, all a power of 2 apart. For example, A is the name given to the pitch on the piano or clarinet with a frequency of 440 Hz as well as 55 Hz, 110 Hz, 220 Hz, 880 Hz, 1760 Hz, and so on. The important thing is the ratio between frequencies, rather than the distance therebetween. For example, 55 Hz to 110 Hz is an octave that happens to span 55 Hz, yet 50 Hz to 100 Hz is also an octave, even though it only covers 50 Hz. But if an orchestra tunes to a different A (as most do nowadays, for example, to middle A=441 Hz or 442 Hz to sound higher and brighter), those frequencies will all change to be multiples/divisors of the new absolute A.

One of the problems inherent to carrying out self-administered hearing tests, is, that while clinical hearing tests are carried out in a controlled environment with a known ambient noise, (e.g. in a sound proof test room), it is virtually impossible to conduct a self-administered hearing test under real life conditions, where ambient noise is not known prior to the beginning of the test, or even worse, it changes from one user to another (as they are subjected to different environmental conditions). Moreover, the ambient noise might even change for a given user during the test itself.

Therefore, it would be beneficial to have a hearing test where emphasis is given to the ratio between frequencies (also referred to as ratios intervals), that will also be adapted to better suit the music world, then hearing tests which are commonly used.

### SUMMARY OF THE DISCLOSURE

The disclosure may be summarized by referring to the appended claims.

It is an object of the present invention to provide a new method for administering a hearing test to an individual.

It is still another object of the present invention to provide a method for modifying audio signals in accordance with hearing capabilities of a user who is listening to the audio signals in accordance with the results obtained while carrying out the novel hearing test of the present invention.

Other objects of the present invention will become apparent from the following description.

According to a first embodiment of the present disclosure, there is provided a method for administering a hearing test to an individual. The method comprises the steps of:
selecting at least one musical melody;
determining a plurality of frequencies at which the at least one musical melody (e.g. a rhythm) or part thereof will be played for the individual;
executing instructions comprised in a computer program to perform steps for administering a hearing test to the individual, wherein the instructions are configured to enable the individual to listen to the at least one musical melody or part thereof at the determined frequencies and at varying intensities;
establishing hearing capabilities of the individual to whom the hearing test has been administered, based on that individual's reactions while listening to the at least one musical melody or part thereof at the determined frequencies and at varying intensities.

According to another embodiment of the present disclosure, the step of determining a plurality of frequencies at which the at least one musical melody or part thereof will be played for the individual, comprises dividing at least part of the at least one musical melody into a plurality of different musical segments.

In other words, the instructions comprised in a computer program may be for example instruction that would cause a sound generating module to generate musical stimuli, wherein the musical stimuli are associated with the selected at least one musical melody or a part thereof.

Optionally, the melody may be generated by sound generating module (e.g. of a personal communication device such as a cellular communication device, etc.) while the set of headphones used by the individual undergoing the hearing test, comprises a processor (e.g. a DSP or even a filter) that may be configured according to some embodiments to enable dividing at least part of the at least one musical melody into a plurality of different musical segments (e.g. based on the audio spectrum comprised in each the plurality of musical segments).

The term “hearing test” as used herein throughout the specification and claims is used to denote a test carrying out to determine the individual's hearing capabilities of one or both ears. The hearing test may be conducted as two separated tests, each for a different individual's ear, or as one test for determining the combined individual's hearing capabilities, when both ears are being subjected simultaneously to the hearing test.

The term “hearing capabilities” as used herein throughout the specification and claims is used preferably but not exclusively to denote gaps that exist between tones that may be heard by an individual who listens to music and the musical tones as they should have been heard by that individual, had he/she had a perfect hearing ability. The gap may be different for different frequencies (or for different range of frequencies) and may be different between the individual's ears. This gap may then be used to allow affecting certain modifications of various musical tones played via the individual's set of headphones in order to decrease these gaps which characterize each specific individual, thereby enhancing the user's experience when listening to a musical creation while using the set of headphones of the present invention.

The term “musical melody” as used herein through the specification and claims, is used to denote a succession of notes forming a distinctive sequence being a pleasing succession or arrangement of sounds. In other words, a rhythmically organized sequence of single tones so related to one another as to make up a particular phrase or idea.

According to another embodiment, the method further comprises a step of selecting the plurality of different musical segments based on psychoacoustic and/or harmonic tones' combinations.

The term “psychoacoustics” as used herein through the specification and claims, is used to denote sound perception. More specifically, it relates to psychological and physiological responses associated with sound (including speech and music). As already explained above, the human ear can nominally hear sounds in the range 20 Hz to 20 kHz. Frequency resolution of the ear is 3.6 Hz within the octave of 1000-2000 Hz. That is, changes in pitch larger than 3.6 Hz can be perceived in a clinical setting. The semitone scale used in Western musical notation is not a linear frequency scale but is a logarithmic one.

The term “harmonic” as used herein through the specification and claims, is used to denote a component frequency of a signal that is an integer multiple of the fundamental frequency, i.e. if the fundamental frequency is f, the harmonics have frequencies 2f, 3f, 4f, . . . etc. The harmonics have the property that they are all periodic at the fundamental frequency, therefore the sum of harmonics is also periodic at that frequency. Harmonic frequencies are equally spaced by the width of the fundamental frequency and can be found by repeatedly adding that frequency. For example, if the fundamental frequency (first harmonic) is 25 Hz, the frequencies of the next harmonics are: 50 Hz (2nd harmonic), 75 Hz (3rd harmonic), 100 Hz (4th harmonic) etc. The untrained human ear typically does not perceive harmonics as separate notes. Rather, a musical note composed of many harmonically related frequencies is perceived as one sound, the quality, or timbre of that sound being a result of the relative strengths of the individual harmonic frequencies.

In accordance with another embodiment, each of the plurality of different musical segments comprises a plurality of tones belonging to a group of frequencies, and wherein a spectrum that comprises all frequencies included in that plurality of groups of frequencies, is substantially larger than a spectrum that matches a frequencies' range of a vocal spectrum.

However, it should be obvious to those skilled in the art that there could be cases where the method provided is applied by using a narrower spectrum (or only parts of the spectrum) than a spectrum that matches a frequencies' range of a vocal spectrum, which may be relevant to certain audience.

By yet another embodiment, the spectrum that comprises all frequencies included in the plurality of groups of frequencies, comprises frequencies belonging to essentially a full musical spectrum.

According to still another embodiment, the method comprises generating the plurality of different musical segments that when taken together constitute the complete musical melody or a substantial part thereof.

In accordance with another embodiment, each of at least two of the plurality of different musical segments constitutes a different spectrum band from the other.

By yet another embodiment, at least two of the plurality of different musical segments are musically similar to each other but are generated (for the individual undergoing the hearing test) at different octaves.

According to another embodiment, at least two of the plurality of different musical segments are musically similar (or even essentially identical) to each other but are generated to the individual undergoing the hearing test, by playing the portion of the musical melody comprised in that segment, by different musical instruments.

According to still another embodiment, at least two of the plurality of different musical segments have a bandwidth of the frequencies comprised in each respective musical segment that is different from a bandwidth of frequencies included in the other musical segment.

In accordance with another embodiment, the step of selecting at least one musical melody, comprises dividing the at least one musical melody or a substantial part thereof into different musical segments, following a determination that the tones that belong to all of the different musical segments represent essentially a full musical spectrum.

By yet another embodiment, the step of determining a plurality of frequencies at which the at least one musical melody or part thereof will be played for the individual, comprises selecting a plurality of musical instruments, where each of the plurality of musical instruments is configured to play the at least one musical melody or part thereof at a pre-determined frequency, being a different frequency from the pre-defined frequencies at which all other selected musical instruments are configured to play the at least one musical melody or part thereof.

In the embodiment described above, as well as throughout the specification and claims, it is described that a plurality of musical instruments is selected, wherein each musical instrument is configured to play the musical melody at a pre-determined frequency (different from the frequencies at which all other selected musical instruments are configured to play). However, as should be understood by any person skilled in the art, when a musical instrument plays a musical melody, that melody is typically not played at a single frequency but rather at a band (or a range) of frequencies which are near the pre-defined frequency. Therefore, the pre-defined frequency as used herein, should be understood to encompass also a band of frequencies which is designated for the comfort of the reader by that single frequency. Typically, the band of frequencies comprises less than or equal to half of the difference extending between a pre-defined selected frequency and the pre-defined proceeding frequency, and less than or equal to half of the difference between extending between a pre-defined selected frequency and the pre-defined preceding frequency. For better understanding the above definition, let us consider the following example, where the pre-defined selected frequency is 2 kHz, the preceding pre-defined frequency is 1 kHz whereas the proceeding pre-defined frequency is 4 kHz. For this example, the use of 2 kHz as being the pre-defined selected frequency, should be understood to encompass in fact all frequencies that are within the range of from 1.5 kHz (half of the difference between 1 kHz and 2kHz) to 3 kHz (half of the difference between 2kHz and 4 kHz).

Furthermore, although the present embodiment refers a to selecting of plurality of musical instruments, each configured to play the musical melody at a different pre-determined frequency from the frequencies at which all other selected musical instruments are configured to play, it should be understood that the embodiment is not restricted to a certain period at which the musical melody will be played to the individual by the different musical instruments. For example, this embodiment also encompasses cases where the hearing test is conducted by using a single musical instrument at a single pre-defined selected frequency, and only at one or more later stages, the hearing test is completed by playing the musical melody to the individual by the other musical instruments, at the other pre-defined frequencies.

According to another embodiment, the pre-defined frequencies are at least two members from a group that consists of 125 Hz, 250 Hz, 500 Hz, 1 kHz, 2 kHz, 4 kHz, 8 kHz and 14 kHz.

By yet another embodiment, at least two different consecutive parts of the melody are each played by a different instrument, while the frequency at which each of the different instruments plays its part (preferably throughout the hearing-test) remains its pre-defined frequency.

In accordance with another embodiment, the at least one melody or parts thereof is played to the individual undertaking the hearing test, each time by a different type of instrument at varying intensity.

According to another embodiment, the method provided further comprises a step of modifying at least part of a melody that will be played to the individual after receiving his/her feedback, while continuing administration of the hearing test.

In accordance with yet another embodiment, the method provided further comprises a step of determining a hearing deviation associated with the individual undergoing the hearing test, wherein the deviation is determined from a mathematical center of an auditory spatial spectrum while that individual is listening to at least part of the musical melody being played in stereo.

According to another aspect of the disclosure there is provided a non-transitory computer readable medium storing a computer program for performing a set of instructions to be executed by one or more computer processors, the computer program is adapted to perform a method for administering a hearing test to an individual that comprises activating a sound generating module to generate musical stimuli for determining the individual's hearing capabilities, wherein the musical stimuli being at least one musical melody (e.g. a rhythm) or part thereof, are played for the individual at a plurality of frequencies.

In accordance with another embodiment, the computer program is executed by a processor comprised in a personal set of headphones.

The term “set of headphones” as used herein throughout the specification and claims is used to denote typically a pair of small loudspeakers designed to be held in place close to a user's ears. They are also known as earspeakers, earphones, etc. Typically, there are three types of headphones—in-ear, on the ear and covering the entire. The alternate in-ear versions are known as earbuds or earphones. Headphones either have wires for connection to a signal source such as an audio amplifier, radio, CD player, portable media player, mobile phone, electronic musical instrument, or have a wireless device, which is used to pick up signal without using a cable. Also, although this term typically relates to a pair of earphones it should be understood to encompass different cases, e.g. a single earphone.

According yet to another aspect of the invention, there is provided an apparatus for administering a hearing test to an individual, comprising:
a storage for storing at least one musical melody;
a sound generating module, configured to play the at least one musical melody or part thereof at pre-determined frequencies and at varying intensities;
a processor configured to:
   determine a plurality of frequencies at which the at least one musical melody or part thereof will be played for the individual during the hearing test;
   execute instructions comprised in a computer program uploaded to the processor, for performing steps for administering a hearing test to the individual by activating said sound generating module;
a user interface configured to receive indications from the individual during administration of said hearing test; and
wherein the processor is further configured to establish hearing capabilities of the individual to whom the hearing test has been administered, based on said indications provided while the individual has been listening to the at least one musical melody or part thereof at the determined frequencies and at varying intensities.

### BRIEF DESCRIPTION OF THE DRAWING

For a more complete understanding of the present invention, reference is now made to the following detailed description taken in conjunction with the accompanying drawings wherein:

FIG. 1—is a flow diagram exemplifying a method carried out in accordance with an embodiment of the present invention; and

FIG. 2—is a flow diagram exemplifying a method carried out in accordance with another embodiment of the present invention.

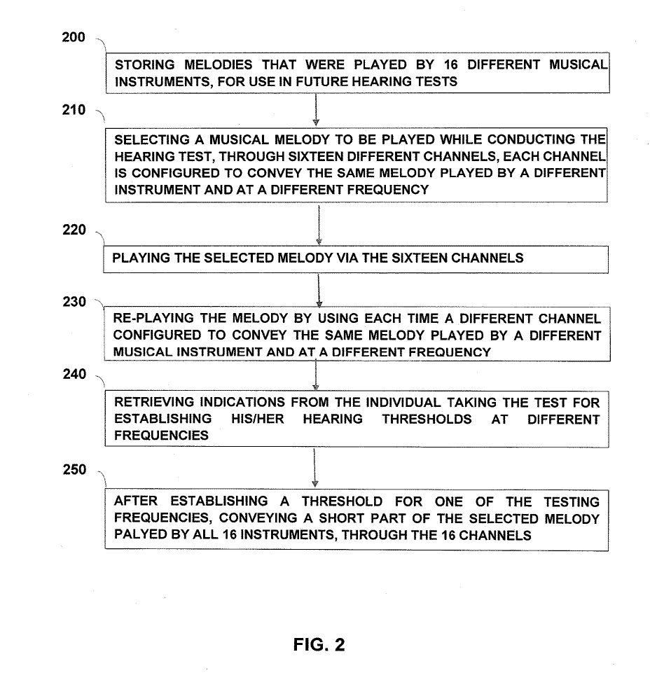

### DETAILED DESCRIPTION

In this disclosure, the term “comprising” is intended to have an open-ended meaning so that when a first element is stated as comprising a second element, the first element may also include one or more other elements that are not necessarily identified or described herein, or recited in the claims.

In the following description, for the purposes of explanation, numerous specific details are set forth in order to provide a better understanding of the present invention by way of examples. It should be apparent, however, that the present invention may be practiced without these specific details.

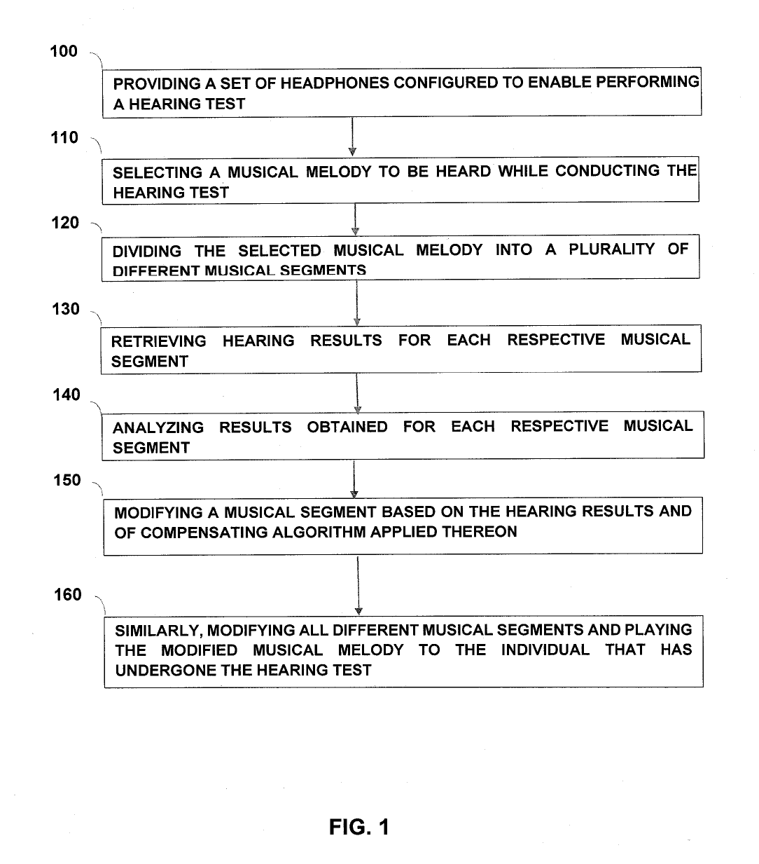

FIG. 1 is a flow diagram being a non-limiting example of a method carried out in accordance with an embodiment of the present invention.

In step 100, a set of headphones is provided. The set of headphones comprises a DSP component, a sound generating module, a controller and a non-transitory computer readable medium (e.g. the DSP, a processor and the like) storing a computer program for performing a set of instructions to be executed by the processor and wherein the computer program is adapted to perform a method in accordance with the present invention for administering a hearing test to an individual.

The hearing test of the present invention differs from standard hearing tests as it is designed to test primarily frequencies that pertain to the comprehension and enjoyment of music, and is thus different from conventional tests that focus on frequencies that make speech more comprehensible for those experiencing certain hearing loss, or deterioration of their hearing capabilities. In other words, conventional clinical hearing tests focus on the vocal spectrum of sound, which is relatively a narrow spectrum. Consequently, if one were to use the outcome of hearing tests for compensation of gaps that exist in an individual's hearing, based on the results of an hearing test he/she had undergone, such a compensation would most likely be non-harmonic and the modified audio signals that will be heard (i.e. the compensated audio signals) would be unpleasant particularly when listening to music.

The terms “a sound generating module” and “a controller” as used herein throughout the specification and claims, should be understood to encompass also any other means suitable for generating sounds in a controlled way, e.g. by using the processor. Thus, all such other means whether in a form of a single means or a combination of means that are used to generate an outcome that is equivalent to that of using the sound generating module and/or controller, i.e. means that are used in a way that they enable generating sounds in a controlled way, are encompassed by the present invention.

Also, it should be noted that the set of instructions to be executed in order to cause the sound generating module to operate, may be loaded onto the set of headphones in accordance with the present invention either during the manufacturing process of the set of headphones, or may be uploaded at any later stage.

Next, one or more musical melodies are selected (step 110) for use in the hearing test. According to one option, one musical melody is pre-selected, whereas according to another option, a number of musical melodies are selected and the one that will eventually be played during the hearing test, may be randomly selected or be selected by the individual taking the test. The selected musical melody (or melodies) is then divided into a plurality of different musical segments (step 120), wherein these segments may be selected based on psychoacoustic and/or harmonic tones' combinations.

Each of the plurality of different musical segments comprises a plurality of tones belonging to a group of frequencies. It should be noted that a spectrum that comprises all frequencies included in the plurality of groups of frequencies, is by far larger than a spectrum that matches a frequencies' range of a vocal spectrum.

Several criteria may be used when making the selection of the plurality of different musical segments. For example, the different musical segments are selected so that when considering a combination of all the groups of frequencies associated with all the musical segments, such a combination will comprise frequencies that cover essentially the full musical spectrum. In other words, the step of dividing the musical melody or a substantial part thereof into different musical segments, involves ensuring that the tones that belong to all of the different musical segments represent essentially the full musical spectrum.

Another selection criterion that may be used is that when the different musical segments are taken together, the result constitutes the complete musical melody or at least a very substantial part thereof.

While making the selection of the different musical segments, it is preferred that at least two of the plurality of musical segments will each constitute a different spectrum band from the other. In addition or in the alternative, at least two of the plurality of musical segments are musically similar to each other but are generated for the individual undergoing said hearing test, at different octaves.

According to another embodiment, at least two of the plurality of musical segments are musically essentially identical to each other but are played by different musical instruments.

Furthermore, it should be noted that the musical segments selected, are not necessarily of the same length. Similarly, the bandwidth of the frequencies comprised in one musical segment is not necessarily the same bandwidth of frequencies included in any of the remaining different musical segments.

The individual undergoing the hearing test listens to the different musical segments played at a varying intensity, e.g., the volume is changed from a very low volume to a higher (louder) volume. When the individual indicates (e.g. by using a user interface connected to the set of headphones, by indicating manually to the technician who conducts the test, or to himself (in case of a self-administered hearing test), that he is able to hear the tone being played, that threshold value of the volume (in dB) is marked as being the result of the individual's hearing test for the band of frequencies included in that musical segment (step 130).

Next (step 140) the results are analyzed and a compensation algorithm is executed thereon. Such a compensating algorithm may include equalization process, in order to determine frequency ranges at which the audio signals will be modified by applying the appropriate compensation for that respective individual. In other words, the compensating algorithm uses predetermined equalizing (EQ) bands and curves to determine the compensation that will be provided to each of the individual's ears. Typically, the compensation (e.g. the frequency ranges in which the modification will be carried out and/or the magnitude of compensation) that will be provided to the audio signal reaching the individual's left ear will be different from the compensation that will be provided to the audio signal reaching the individual's right ear, based on the results obtained.

It should be noted that determining the required compensation is a rather complex process, which should take into account different frequencies and bands (in terms of the range of frequencies affected) and is preferably used to generate a smooth and enjoyable equalized curve for each ear. For example, if a user has a 3 db drop at around 500 Hz, it might be more musically pleasing to enhance the frequencies around 500 Hz by 2.5 db through a broadband curve extending from 450 Hz to 570 Hz, rather than just bumping the 500 Hz drop by 3 db. Another example could be to reduce the level of few frequencies around the 500 Hz frequency instead of bumping the signal at the 500 Hz frequency.

Optionally, once the preferred compensation has been determined for a musical segment for the individual undergoing the test, that musical segment is modified according to the determination of the compensation required for the musical segment, and the modified musical segment (i.e. after the adjustment of the audio signals contained therein has been made), may be played as part of the continued test (step 150). This type of operation may then be repeated for other musical segments so that eventually towards the end of the hearing test, the individual taking the test may be able to listen to the whole musical melody that comprises the various musical segments in their modified form (step 160).

The human brain is adapted to compensate for differences existing between the hearing capabilities of the individual's ears in order to solve spatial orientation problems. Thus, according to an embodiment of the invention, the method may optionally further comprise the steps of:
a. Forwarding a stereo audio signal at a given frequency or at a range of frequencies towards both ears of the individual undergoing the hearing test;
b. Allowing the individual to indicate a point at which the stereo sound is heard to him as being in balance between his left and right ears, i.e. the middle of the auditory spatial spectrum;
c. Determining the deviation that exists between the point indicated by the individual to be the middle of the auditory spatial spectrum and the physical center between the individual left and right ears; and
d. Repeating the above steps a to c while using different frequencies/different ranges of frequencies, to enable generating a map of such deviations as a function of the different frequencies/ranges of frequencies, thereby enabling to provide that individual with audio compensations while listening to audio signals being played in a stereo mode.

Optionally, data resulting from the determination made by the compensation algorithm, may be stored (e.g. as a firmware at the DSP comprised within the headphones). This step allows modifying and adjusting audio signals for the specific individual when he/she decides to use the set of headphones for listening to music. When that happens, the data resulting from the compensation algorithm that had been stored, would be provided to two customized equalizers (for the L/R ears) in such a way that the audio signals are modified in each of the two channels, accordingly. The individual listening experience when listening to a musical creation (different from the one used in the hearing test) would therefore be enhanced dramatically, after implementing the modifications and adjustments described above that match his own hearing capabilities.

Optionally, but not necessarily, after completing the compensation process described above for both ears of the individual, he/she is able to be subjected to a test where he/she would listen to a piece of music with and without the modifications, to enable determining whether a different compensation should be applied. After the individual has indicated that the compensation results are satisfactory, the equalization data (i.e. Frequency, Volume and “Q” value) are forwarded to the DSP comprised within the headphones, with or without changing the firmware embedded at the DSP, and the customized equalization data is stored at the headphones.

The value of parameter “Q” referred to above, relates to the angle and scope of the gain given for each frequency band. It may be very sharp and bell shaped, thus affecting only a rather narrow range of frequencies, or on the other hand it may be quite flat and large, thus affecting a larger range of frequencies.

Let us now consider an example of another embodiment according to the present invention for administering a hearing test to an individual, where the hearing test is based on subjecting the individual to a piece of music, rather than subjecting the individual to a series of beep tones.

In this example, the hearing test was conducted by applying melodies that had been played by 16 different musical instruments, recorded and stored to be played during future hearing tests via 16 respective channels (step 200). The melodies played by all of the musical instruments in this test, were all the same melody. For testing each of the individual ears, a set of 8 frequencies was used that included the following pre-defined frequencies 125 Hz, 250 Hz, 500 Hz, 1 kHz, 2 kHz, 4 kHz, 8 kHz and 14 kHz. In other words, for each of the individual ears, a melody that was generated by a different musical instrument was played at a different, pre-defined frequency, a frequency that was different from the frequencies at which the other musical instruments played that melody.

First, the melody was played via all 16 channels. The melody was selected so that when it is played via all these channels, the result heard by the individual whose hearing is being tested, is a coherent musical piece which is pleasant to listen to (step 210).

The melodies that were applied in this example test, were recorded while playing the following musical instruments at the respective frequencies:

For the left ear test:
For the frequency of 125 Hz: a live kick drum;
For the frequency of 250 Hz: a live bass guitar;
For the frequency of 500 Hz: an electric guitar;
For the frequency of 1 kHz: a snare drum;
For the frequency of 2 kHz: vocals—lead;
For the frequency of 4 kHz: bongos;
For the frequency of 8 kHz: a tambourine; and
For the frequency of 14 kHz: a ride cymbal.

For the right ear test:
For the frequency of 125 Hz: an electronic bass drum;
For the frequency of 250 Hz: a synth bass;
For the frequency of 500 Hz: a Hammond organ;
For the frequency of 1 kHz: claps;
For the frequency of 2 kHz: backing vocals;
For the frequency of 4 kHz: a brass instrument;
For the frequency of 8 kHz: a high-hat; and
For the frequency of 14 kHz: a shaker.

As will be appreciated by those skilled in the art, while the melody is being played during this part of the test, it could be played simultaneously via a number of channels (i.e. the recorded melody that had been played by a number of instruments, is played to the individual simultaneously). However, it should be noted that irrespective if the melody is played during this step in one channel (i.e. by a single musical instrument), in a number of channels (i.e. by a plurality of musical instruments), or any combination thereof, the melody played in each channel is always played at the pre-defined frequency for that specific musical instrument.

Thus, as will be appreciated by those skilled in the art, the method exemplified herein enables the use of many different musical melodies, while still adhering to the principles described hereinabove.

After the individual has listened to the musical melody played by the various musical instruments (step 220), the test continues (step 230) by playing the melody each time via a single channel. In other words, the individual who is undergoing the hearing test, hears at this step, each musical instrument playing the melody by itself (solo), in a decreasing intensity (e.g. applying 5 dB decreases each time) until the individual indicates that he/she does not hear the melody any longer, thereby establishing the respective thresholds for each of the musical instruments (i.e. for each of the frequencies) (step 240). Therefore, when testing each ear of the individual, that individual listens according to this example, nine times to the same melody (once when all the instruments participate, and eight other time when each instrument plays the melody at the pre-defined frequency assigned to that musical instrument.

Notwithstanding the above, for better understanding this example, it should be noted that although the same musical melody is played by each of the musical instrument, still, it may sound differently to the individual undergoing the hearing test, as each musical instrument may be used to play only part of the musical melody, not necessarily the same part as that (those) played by the one or more other musical instruments.

Optionally, when the individual indicates that the melody played by a certain instrument becomes inaudible, i.e. after defining the threshold associated with the respective frequency, an excerpt of the melody played by all 16 instruments, is played for a short while to the individual (step 250) and then the melody would be played once again, this time by a different musical instrument (i.e. at a different frequency).

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention in any way. The described embodiments comprise different features, not all of which are required in all embodiments of the invention. Some embodiments of the present invention utilize only some of the features or possible combinations of the features. For example, although the description above relates to carrying out the method provided by components comprised within the set of headphones, still, it should be appreciated that the method may be exercised by having an intermediate device comprising the DSP described above which will be fitted between any headphone jack and the headphones themselves (any set of headphones). This will allow users to improve their sound listening experience by implementing the method described herein, without having to change their set of headphones, or without having to buy any particular set of headphones.

Variations of embodiments of the present invention that are described and embodiments of the present invention comprising different combinations of features noted in the described embodiments will occur to persons of the art. The scope of the invention is limited only by the following claims.

1. A method for administering a hearing test to an individual which comprises the steps of:
selecting at least one musical melody;
determining a plurality of frequencies at which the at least one musical melody or part thereof will be played for the individual;
executing instructions comprised in a computer program to perform steps for administering a hearing test to the individual, wherein the instructions are configured to enable the individual to listen to the at least one musical melody or part thereof at the determined frequencies and at varying intensities;
establishing hearing capabilities of the individual to whom the hearing test has been administered, based on that individual's reactions while listening to the at least one musical melody or part thereof at the determined frequencies and at varying intensities.

2. The method of claim 1, wherein the step of determining a plurality of frequencies at which the at least one musical melody or part thereof will be played for the individual, comprises dividing at least part of the at least one musical melody into a plurality of different musical segments.

3. The method of claim 2, further comprising a step of selecting the plurality of different musical segments based on psychoacoustic and/or harmonic tones' combinations.

4. The method of claim 2, wherein each of the plurality of different musical segments comprises a plurality of tones belonging to a group of frequencies, and wherein a spectrum that comprises all frequencies included in that plurality of groups of frequencies, is substantially larger than a spectrum that matches a frequencies' range of a vocal spectrum.

5. The method of claim 4, wherein the spectrum that comprises all frequencies included in the plurality of groups of frequencies, comprises frequencies belonging to essentially a full musical spectrum.

6. The method of claim 2, comprising generating the plurality of different musical segments that when taken together constitute the complete musical melody or a substantial part thereof.

7. The method of claim 2, wherein each of at least two of the plurality of different musical segments constitutes a different spectrum band from the other.

8. The method of claim 2, wherein at least two of the plurality of different musical segments are musically similar to each other but are generated at different octaves.

9. The method of claim 2, wherein at least two of the plurality of different musical segments are musically similar to each other, but are generated to the individual undergoing the hearing test by playing the portion of the musical melody comprised in that segment with different musical instruments.

10. The method of claim 2, wherein at least two of the plurality of different musical segments have a bandwidth of the frequencies comprised in each respective musical segment that is different from a bandwidth of frequencies included in the other musical segment.

11. The method of claim 2, wherein the step of dividing the at least one musical melody or a substantial part thereof into different musical segments, follows a determination that the tones that belong to all of the different musical segments represent essentially a full musical spectrum.

12. The method of claim 1, wherein the step of determining a plurality of frequencies at which the at least one musical melody or part thereof will be played for the individual, comprises selecting a plurality of musical instruments, where each of the plurality of musical instruments is configured to play the at least one musical melody or part thereof at a pre-determined frequency, being a different frequency from the pre-defined frequencies at which all other selected musical instruments are configured to play the at least one musical melody or part thereof.

13. The method of claim 12, wherein the pre-defined frequencies are at least two members from a group that consists of 125 Hz, 250 Hz, 500 Hz, 1 kHz, 2 kHz, 4 kHz, 8 kHz, 14 kHz.

14. The method of claim 12, wherein at least two different consecutive parts of the melody are each played by a different instrument, while the frequency at which each of the different instruments plays its part throughout the hearing-test remains its pre-defined frequency.

15. The method of claim 12, wherein the at least one melody or parts thereof is played to the individual undertaking the hearing test, each time by a different type of instrument at varying intensities.

16. The method of claim 1, further comprising a step of modifying at least part of a melody that will be played to the individual after receiving his/her feedback, while continuing administration of the hearing test.

17. The method of claim 1, further comprising a step of determining a hearing deviation associated with the individual undergoing the hearing test, wherein the deviation is determined from a mathematical center of an auditory spatial spectrum while that individual is listening to at least part of the musical melody being played in stereo.

18. A non-transitory computer readable medium storing a computer program for performing a set of instructions to be executed by one or more computer processors, the computer program is adapted to perform a method for administering a hearing test to an individual that comprises activating a sound generating module to generate musical stimuli for determining the individual's hearing capabilities, wherein the musical stimuli being at least one musical melody or part thereof, are played for the individual at a plurality of frequencies.

19. The computer program of claim 18, configured to be executed by a processor comprised in a personal set of headphones.

20. An apparatus for administering a hearing test to an individual, comprising:
a storage for storing at least one musical melody;
a sound generating module, configured to play the at least one musical melody or part thereof at pre-determined frequencies and at varying intensities;
a processor configured to:
determine a plurality of frequencies at which the at least one musical melody or part thereof will be played for the individual during the hearing test;
execute instructions comprised in a computer program uploaded to the processor, for performing steps for administering a hearing test to the individual by activating said sound generating module;
a user interface configured to receive indications from the individual during administration of said hearing test; and
wherein the processor is further configured to establish hearing capabilities of the individual to whom the hearing test has been administered, based on said indications provided while the individual has been listening to the at least one musical melody or part thereof at the determined frequencies and at varying intensities.
